(19) United States
(12) Patent Application Publication
(10) Pub. No.: US 2013/0023786 A1
(43) Pub. Date: Jan. 24, 2013

(54) PREDICTING URINATION

(71) Applicants: Murali Mani, Chappaqua, NY (US); Ramon Quido Erkamp, Yorktown Heights, NY (US)

(72) Inventors: Murali Mani, Chappaqua, NY (US); Ramon Quido Erkamp, Yorktown Heights, NY (US)

(73) Assignee: KONINKLIJKE PHILIPS ELECTRONICS N.V., EINDHOVEN (NL)

(21) Appl. No.: 13/580,092

(22) PCT Filed: Mar. 31, 2011
(86) PCT No.: PCT/IB11/51374

§ 371 Date: Aug. 20, 2012

Related U.S. Application Data
(60) Provisional application No. 61/321,903, filed on Apr. 8, 2010.

(51) Int. Cl. A61B 5/03; A61B 5/00

(52) U.S. Cl. 600/552; 600/561

(57) ABSTRACT

An apparatus comprising a sensing unit configured to predict a release of urine using one or more prediction parameters, detect an actual release of urine and adapt at least one of the one or more prediction parameters based on the actual release of urine to increase prediction accuracy.

### FIELD OF THE INVENTION

The invention relates to a sensing apparatus and a method for predicting urination. Particularly, but not exclusively, the invention relates to predicting when urination will occur and providing an alert based on the prediction.

### BACKGROUND TO THE INVENTION

An unexpected release of urine due to a lack of bladder control can be unpleasant and distressing for the person involved. It is therefore desirable that the urine is dealt with as quickly as possible and that the frequency of such occurrences is kept to a minimum.

A previous solution has been to provide a wearable device which reacts to a release of urine by emitting an audible alert. The alert allows a parent or carer to deal with the urine quickly, thereby preventing any potentially unhygienic effects and minimizing discomfort felt by the person involved. However, the reactive nature of such a device fails to prevent the actual release of the urine.

Another solution has been to provide a device which measures a state of fullness of the bladder and provides a signal when the measurement exceeds a threshold value. However, different bladder sizes and the varying nature of human bodies make such devices inaccurate.

It is therefore preferable that a better solution be found to prevent unexpected releases of urine due to urinary incontinence.

### SUMMARY OF THE INVENTION

According to the invention, there is provided a sensing apparatus configured to predict a release of urine using one or more prediction parameters, detect an actual release of urine and adapt at least one of the one or more prediction parameters based on the actual release of urine to increase prediction accuracy.

The sensing apparatus may be configured to adapt the at least one prediction parameter based on a difference between the predicted release of urine and the actual release of urine.

The sensing apparatus may be configured to predict the release of urine by predicting a time at which the release of urine will occur, detect the actual release of urine by detecting a time at which the actual release of urine occurs and adapt the at least one prediction parameter based on a time difference between the predicted time of urine release and the detected time of urine release.

The sensing apparatus may be configured to make intermittent estimates of a state of fullness of a bladder and to predict the release of urine using the state of fullness estimates and the prediction parameters.

The sensing apparatus may be configured to enter a power-saving mode between the intermittent state of fullness estimates.

The sensing apparatus may be configured to estimate the state of fullness of the bladder by emitting an ultrasound wave towards the bladder, detecting an ultrasound wave reflected back from a wall of the bladder and analyzing the reflected ultrasound wave to estimate the state of fullness of the bladder.

The sensing apparatus may comprise a plurality of ultrasound transducer elements for emitting and detecting the ultrasound waves, driving and receiving circuitry configured to respectively cause the ultrasound transducer elements to emit the ultrasound waves and to detect the ultrasound waves in the ultrasound transducer elements and a controller configured to estimate the state of fullness of the bladder and to predict the release of urine based on the estimated state of fullness of the bladder and the prediction parameters.

The ultrasound transducer elements may comprise one or more piezoelectric crystals or CMUTS.

The sensing apparatus may be configured to adapt at least one of the one or more prediction parameters based on the position of the sensing apparatus relative to a bladder.

The sensing apparatus may comprise a sensing unit which is mountable over a bladder on the exterior of a body.

The apparatus may additionally comprise an external unit configured to communicate with the sensing unit for predicting the release of urine.

The sensing apparatus may comprise an alarm unit configured to provide information as to when urine is predicted to be released.

The sensing apparatus may comprise one or more indicators configured to indicate when the sensing apparatus is correctly located in a position overlapping a bladder.

The indicators may comprise one or more LEDs configured to provide a visible indication as to whether the sensing apparatus is positioned correctly over the bladder.

According to the invention there is also provided an item of clothing comprising at least part of the sensing apparatus, for example the sensing unit. The item of clothing may comprise a diaper

According to the invention, there is also provided a method for warning of a release of urine comprising predicting a release of urine using one or more prediction parameters, detecting an actual release of urine and adapting at least one of the one or more prediction parameters based on the actual release of urine to increase prediction accuracy.

Predicting the release of urine may comprise predicting a time at which the release of urine will occur, detecting the actual release of urine may comprise detecting a time at which the actual release of urine occurs, and adapting the at least one prediction parameter may comprise adapting the at least one prediction parameter based on a time difference between the predicted time of urine release and the detected time of urine release.

According to the invention there is also provided a computer program which, when executed by a processor, causes the processor to perform the method.

These and other aspects of the invention will be apparent from and elucidated with reference to the embodiments described hereinafter.

Embodiments of the invention will now be described, by way of example, with reference to the accompanying figures in which:

### BRIEF DESCRIPTION OF THE FIGURES

FIG. 1 is a schematic illustration of a sensing unit for predicting a release of urine.

FIG. 2 illustrates a possible location of the sensing unit in a diaper and an external unit for communicating with the sensing unit.

FIG. 3 is a perspective illustration of a matrix of nine piezoelectric crystals for emitting and detecting ultrasound waves to detect characteristics of a bladder.

FIG. 4 is a schematic illustration of a control unit for predicting a release of urine and alerting a user based on ultrasound waves detected at the piezoelectric crystals.

FIG. 5 is flow diagram of an example of a method of predicting a release of urine.

### DESCRIPTION OF EMBODIMENTS

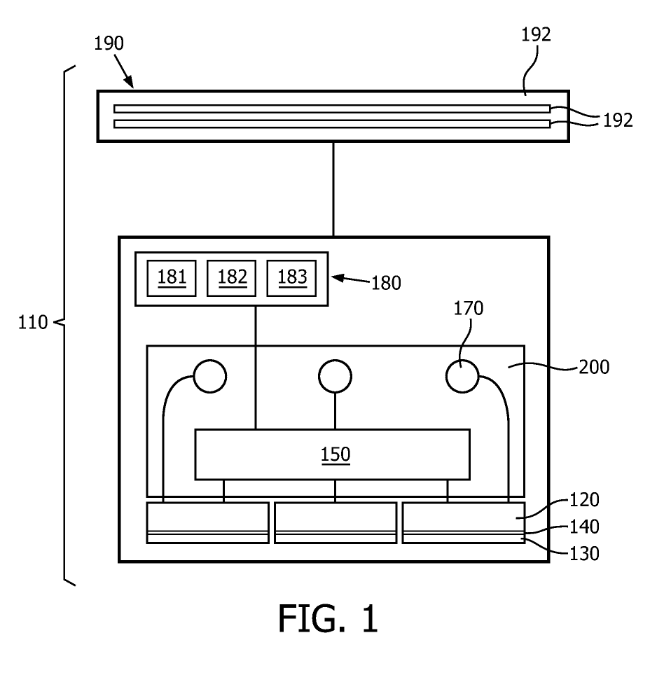

FIG. 1 illustrates an example of a sensing apparatus 100 for predicting a release of urine. The apparatus is described below in terms of predicting a release of urine from the bladder of a human being. However, the sensing apparatus 100 could alternatively be used for predicting a release of urine from the bladder of an animal such as a pet mammal.

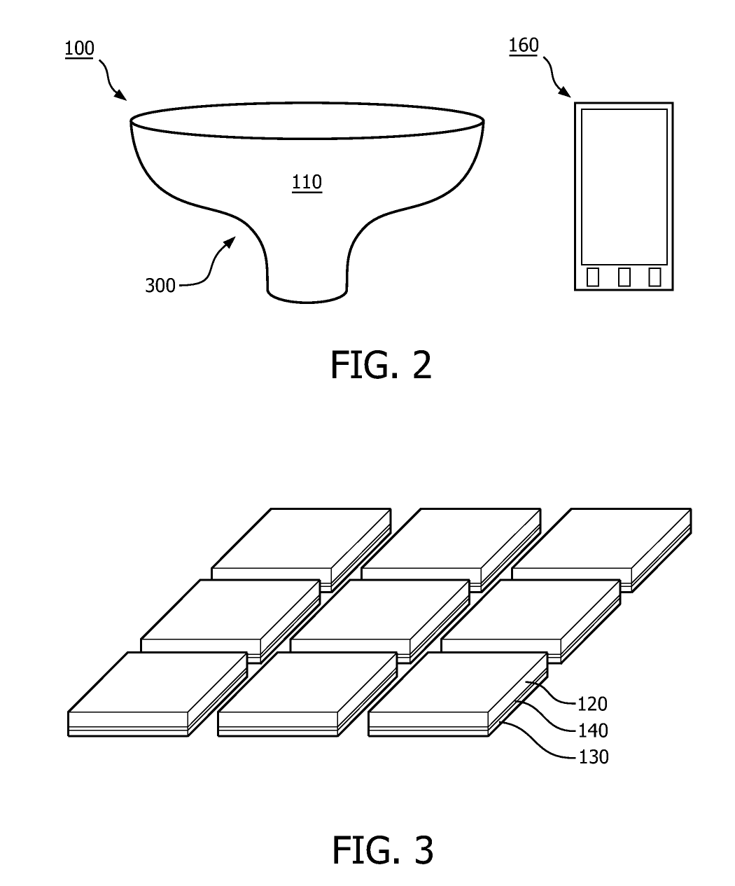

The sensing apparatus 100 comprises a sensing unit 110 which can be positioned over the bladder of a human body. For example, the sensing unit 110 can be integrated into or fixed to underwear, a diaper 300 or another suitable item of clothing so as to be held against the skin at a position which overlaps the bladder. This is shown in FIG. 2. Alternatively, the sensing unit 110 can be provided with a belt for attaching around the waist. It will be appreciated that other suitable means of attachment are equally possible.

The sensing unit 110 comprises at least one transducer 120 for estimating the state of fullness of the bladder using ultrasound. For example, as shown in FIG. 3, the sensing unit 110 may comprise an array of ultrasound transducer elements 120. The ultrasound transducer elements 120 may comprise a square matrix of nine substantially square piezoelectric crystals 120 each having sides of approximately 10 mm, a depth of approximately 0.2 mm and a centre frequency of approximately 10 MHz. A gap of approximately 2 mm is provided between neighboring crystals 120 in the matrix.

The sensing apparatus 100 is discussed below using the piezoelectric crystal example shown in FIG. 3. However, the number, dimensions, spacing and centre frequencies of the ultrasound transducer elements 120 are not limited to this example. For instance, Capacitive Micromachined Ultrasonic Transducers (CMUTS) can be used in addition or as an alternative to piezoelectric crystals. Alternatively, other types of transducer 120 can be provided. An example is a transducer configured to detect electrical signals in the skin of the user which are indicative of the state of fullness of the bladder.

Optionally, a corresponding array of acoustic lenses 130 is coupled to a major face of the array of ultrasound transducer elements 120. In FIG. 3, a square matrix of nine acoustic lenses 130 is provided such that an acoustic lens 130 is provided on a square face of each of the nine piezoelectric crystals 120 described above. The dimensions of the major faces of the acoustic lenses 130 may substantially match those of the crystals 120. A gap of 2 mm may be provided between the lenses 130 in a similar manner to that described above for the crystals 120. It will be understood that the size of this gap can alternatively be larger or smaller, for example in order to match the size of the gap between the crystals 120. As can be seen in FIG. 3, all of the acoustic lenses 130 are provided on the same side of the array of crystals 120. An acoustic matching layer 140 can be provided between the crystals 120 and the acoustic lenses 130. A suitable acoustic matching layer 140 comprises a sheet of Kapton.

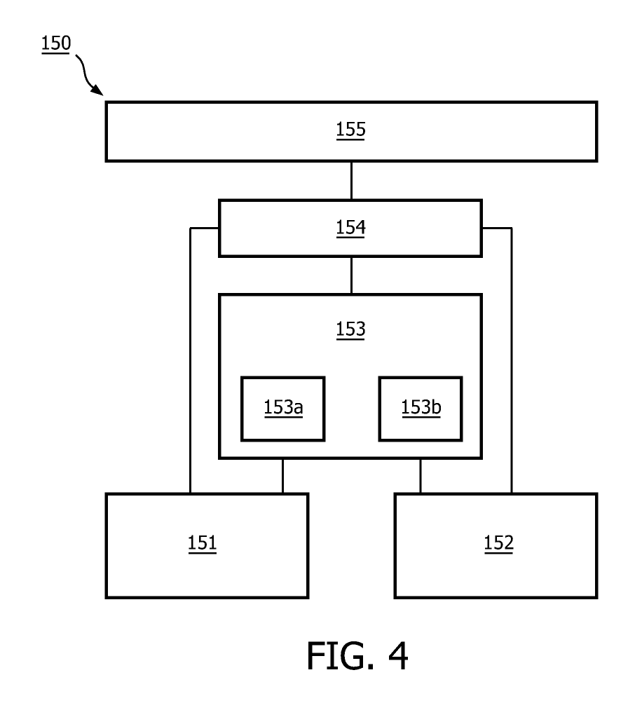

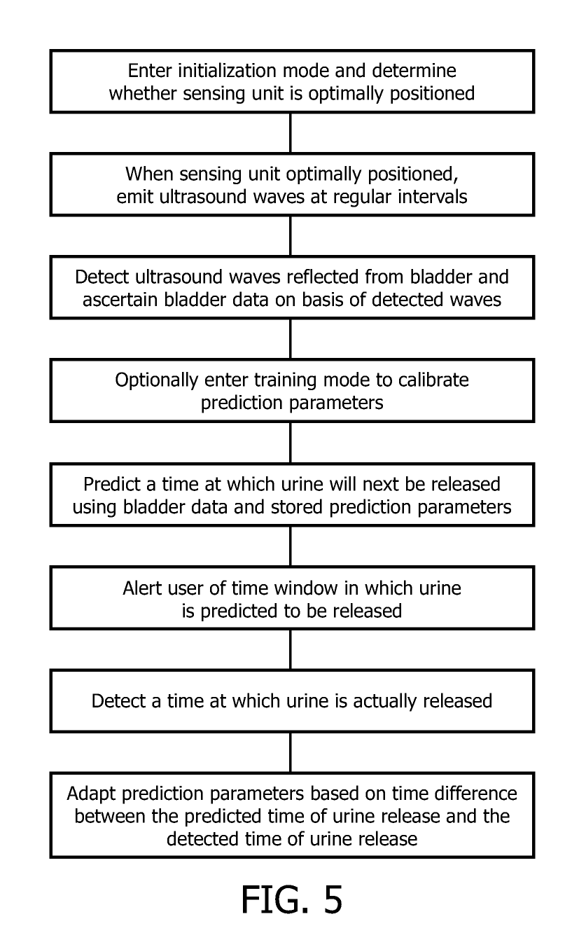

A control unit 150 is electrically coupled to a major face of the transducer array 120, for example to the opposite face of the transducer array 120 to the acoustic lenses 130. This is exemplified in FIG. 1, in which the control unit 150 is electrically coupled to a major face of each of the nine piezoelectric crystals 120. The operation of the control unit 150 for predicting a release of urine is described below in relation to FIG. 4. An example method for predicting a release of urine is also described below and is illustrated in FIG. 5.

Referring to FIG. 4, the control unit 150 comprises electronic driving circuitry 151 and receiving circuitry 152 connected to apply and receive electrical signals at each crystal 120 via a pair of electrodes. The electrodes on one side of the crystals 120 may be provided as metalized electrodes on the acoustic matching layer 130, whilst the electrodes on the other side of the crystals 120 may extend directly from the driving and receiving circuitry 151, 152.

The control unit 150 further comprises a microcontroller 153, which includes a processing unit 153a and a memory 153b. The microcontroller 153, receiving circuitry 152 and driving circuitry 151 may all be powered by a battery unit 154 which is preferably a compact re-chargeable battery 154 in the sensing unit 110. An inductive coil 155 is provided at a suitable location in the sensing unit 110, for example around the perimeter of the unit 110, for inductively charging the battery 154 from an external energy source.

The control unit 150 may be comprised completely in the sensing unit 110 as shown in FIG. 1. However, as an alternative, the control unit 150 may be partly comprised in a separate, external unit 160. This is shown in FIG. 2. For example, the microcontroller 153, processing unit 153a and memory 153b may be provided in an external handheld device 160 such as a mobile telephone, smart telephone, laptop computer or Personal Digital Assistant (PDA). If the microcontroller 153 is provided in the external unit 160, it is configured to communicate with the remaining elements of the control unit 150, such as the driving and receiving circuitry 151, 152, via a wireless communication link between the external unit 160 and the sensing unit 110. The communication link can be implemented using Bluetooth and is facilitated by providing each of the external unit 160 and the sensing unit 110 with a suitable Bluetooth transceiver. If the wireless link between the sensing unit 110 and the external unit 160 is interrupted, for example by the sensing unit 110 and the external unit 160 being too far apart, the external unit 160 and/or the sensing unit 110 may be configured to activate an alarm as a warning to the user of the apparatus 100 or their carer.

If elements of the control unit 150 are provided in an external unit 160 as described above, power for those elements is provided by a battery within the external unit 160. This reduces power consumption in the sensing unit 110 and therefore allows the battery unit 154 in the sensing unit 110 to power the sensing unit 110 for longer without requiring to be recharged.

Referring again to FIG. 1, the driving circuitry 151 is configured to apply voltage signals to the transducer elements 120 via the electrodes. The voltage signals applied by the driving circuitry 151 are selected so as to cause the transducer elements 120 to emit ultrasound waves which, when the sensing unit 110 is correctly positioned over the bladder, are directed towards the bladder walls. If piezoelectric crystals 120 are used as described above, the ultrasound waves propagate towards the bladder as a straight beam (with a crystal shaped cross section) for several centimeters before slowly diverging outwards. The emitted sound waves may thus be described as “A-lines”. Reflections of the ultrasound waves from the bladder walls are detected at the crystals 120 and used to estimate the state of fullness of the bladder, as is described below. The voltage signals applied by the driving circuitry 151 are controlled by the microcontroller 153, for example based on parameters stored in the microcontroller's memory 153b. If the microcontroller 153 is provided in the external unit 160 described above, the microcontroller 153 controls the application of the voltage signals via instructions sent via the wireless communication link.

The receiving circuitry 152 is configured to detect voltage signals experienced in the piezoelectric crystals 120 via the electrodes. The detected voltage signals are due to vibrations in the piezoelectric crystals 120 caused by ultrasound waves reflected back from the front and rear walls of the bladder and tissues surrounding the bladder. The microcontroller 153 is configured to analyze the voltage signals detected in the piezoelectric crystals 120 to calculate the current size characteristics of the bladder, which in turn can be used by the microcontroller 153 to estimate the state of fullness of the bladder. This is described in more detail below. If the microcontroller 153 is provided in the external device 160, information describing the voltage signals detected in the piezoelectric crystals 120 is transmitted to the microcontroller 153 via the wireless communication link.

The properties of the voltage signal detected in each particular crystal 110 are related to the distance between the front and rear walls of the bladder at the location of each crystal 110. The voltage signal may for example indicate the time difference between the receipt of an ultrasound wave reflected from the front wall of the bladder and the receipt of an ultrasound wave reflected from the rear wall of the bladder. The time difference can be used to directly estimate the state of fullness of the bladder (where a longer time difference indicates a fuller bladder state). Alternatively, the state of fullness can be estimated by calculating the distance between the front and rear walls of the bladder using the time difference referred to above and the speed of the ultrasound wave. The calculation can be based on the low echogenicity of urine versus bladder tissue and can, for example, take place in the microcontroller 153. As a further alternative, the state of fullness of the bladder can be estimated by estimating the volume of the bladder based on the time difference referred to above (or calculated distance) detected at each crystal 120 in the transducer array.

In this way the state of fullness of the bladder can be tracked over a specified time period or at regular intervals by regularly emitting and detecting ultrasound waves.

As referred to above, the sensing unit 110 should optimally be worn at a location directly overlapping the bladder so that ultrasound waves emitted from each of the transducers 110 are reflected back to the transducer array 110 from the walls of the bladder. Referring to FIG. 1, to assist with positioning the unit 100, the sensing unit 110 can optionally comprise an LED 170 or other suitable indicator 170 at the location of each of the piezoelectric crystals 120. The LEDs 170 are configured to indicate to the user or a carer when the unit 110 is in the correct position. For instance, during placement of the sensing unit 110, the control unit 150 may act in an initialization mode in which ultrasound waves are continuously emitted from the piezoelectric crystals 120. The voltage signals detected in the piezoelectric crystals 120 due to reflected ultrasound waves are likewise continuously monitored and communicated to the user or carer via the LEDs or other indicators 170 to indicate when the unit 110 is correctly positioned. One implementation is to provide a first color (e.g. red) LED at the location of each of the eight perimeter crystals 120 in the array and a second color (e.g. green) LED at the location of the centre crystal 110. When the voltage signal detected at the centre crystal 110 indicates that the distance between the front and rear bladder walls is greater than the distance between the front and rear walls at the perimeter crystals 120, the green LED 170 is switched on by the microcontroller 153 to tell the user or carer that the sensing unit 110 is optimally positioned. In a similar fashion, if the voltage signal (or lack of voltage signal) detected at any of the perimeter crystals 120 indicates that ultrasound waves are not being reflected back from the bladder walls at that location, the red LED 170 for that crystal 110 is switched on by the microcontroller 153 to tell the user or carer that the sensing unit 110 is not optimally positioned.

The LEDs 170 can be provided at an exterior surface of the sensing unit 110. Alternatively, they can be embedded inside a translucent sealant material 200 in the sensing unit 110. The sealant material 200 is discussed in more detail below. Power for the LEDs 170 can be provided by the battery unit 154 in the control unit 150.

As a further alternative, indicators 170 representing each crystal location can be provided on a display screen of the external unit 160. If this is the case, information describing the voltage signals detected at the crystals 110 is passed between the receiving circuitry 152 and the microcontroller 153 via the wireless link. The microcontroller 153 controls the indicators 170 to aid the user or carer in positioning the sensing unit 110 by displaying an image on the display screen of the external unit 160 which corresponds to the LED pattern described above. This would save the cost and power consumption associated with providing the LEDs 170 in the sensing unit 110.

Once the sensing unit 110 has been correctly positioned on the body, for example using the initialization mode described above, the driving circuitry 151 causes the piezoelectric crystals 120 to emit ultrasound waves towards the bladder at regular intervals. A suitable interval can be every sixty seconds. Different intervals can also be used. In the time periods between the emission and detection of ultrasound waves, the sensing unit 110 is preferably configured to enter a power-saving mode in which power is not supplied to the driving and receiving circuitry 151, 152.

Ultrasound signals reflected from the bladder walls are detected by the receiving circuitry 152 and data indicating the state of fullness of the bladder is recorded together with a timestamp indicating the time at which the recording was taken. The data can include the distance between the front and rear bladder walls detected at each of the crystals 120 (or a time difference representing the distance, as described above), an estimate of absolute volume for the bladder and the increase/decrease in the estimated volume of the bladder since the time of the last estimate. The data is stored in the memory 153b of the microcontroller 153, although it will be appreciated that alternative storage means are equally possible. The stored data can be used for predicting when the next release of urine will occur, as described below.

Using the data collected from the detected voltage signals in the piezoelectric crystals 120, the control unit 150 is configured to predict the time at which urine will next be released from the bladder. More specifically, the control unit 150 is configured to predict the time of the next release of urine on the basis of the collected bladder fullness data and one or more prediction parameters stored in the memory 153b of the microcontroller 153. The prediction parameters are initially preloaded into the memory 153b and may include, for example, threshold values indicative of an imminent release of urine such as a threshold bladder volume or a threshold time difference. In one implementation, an algorithm configured to predict the time of the next release of urine using the bladder fullness data (e.g. by directly inputting the voltage signals detected by the receiving circuitry 152) and the prediction parameters is stored in the memory 153b of the microcontroller 153. The algorithm can be model based or, for example, can be based on neural nets or fuzzy logic. The algorithm is preferably implemented as a computer program which, when executed by the processing unit 153a of the microcontroller 153, causes the processing unit 153a to output a prediction as to when urine will next be released. The predicted urine release time can be stored in the memory 153b of the microcontroller 153 and adjusted each time another set of data indicating the state of fullness of the bladder is collected.

If the control unit 150 is provided in the external unit 160 as discussed above, the algorithm can be comprised in an application loaded into the memory of the external unit 160.

The sensing apparatus 100 further comprises an alarm unit 180 which is electrically coupled to the control unit 150 and configured to output an alarm warning the user or carer that the predicted urine release time is approaching. A suitable activation time for the warning alarm could be five minutes prior to the predicted urine release time. The alarm unit 180 may, for example, comprise a loudspeaker 181 and/or light 182 configured to respectively output an audible or visible alarm to warn to the user or carer that a release of urine is expected. The loudspeaker 181 may optionally comprise one or more headphones so that the audible alarm is relatively discreet. Additionally or alternatively, the alarm unit 180 may comprise a discreet vibration device 183.

The alarm unit 180 can be provided in the sensing unit 110 as shown in FIG. 1. In this case, power for the alarm unit 180 can be provided by the battery 154 in the sensing unit 110, or by a separate power supply source such as secondary battery in the alarm unit 180. Alternatively, the alarm unit 180 can be provided in the external unit 160 and powered by a battery in the external unit 160. In this case, the microcontroller 153 controls the alarm unit 180 using signals received from the receiving circuitry 152 via the wireless link.

The sensing unit 110 also comprises a urine sensor 190 to detect an actual release of urine from the bladder. For example, the urine sensor 190 may comprise a flexible strip of material 191 having one or more electrically conductive elements 192 extending between opposite ends of the strip 191. This is shown in FIG. 1. When the sensing unit 110 is positioned correctly on the user's body, the strip 191 is located close to the user's genitals so that any urine released from the genitals leaks onto the strip 191 and is detected. If conductive elements 192 are used, the detection of urine may be made by detecting a significant drop in the electrical impedance of the conductive elements 192. The determination of whether urine has been released could, for example, be made at the microcontroller 153 by electrically coupling the conductive elements 192 to the control unit 150. Optionally, the material of the strip 191 surrounding the conductive elements 192 may be highly absorbent to urine so as to reliably convey released urine to the conductive elements 192 for detection.

When a release of urine is detected by the urine sensor 190, data regarding the urine release is recorded by the control unit 150. The stored data includes the time at which the urine was detected by the urine sensor 190, and may include other data such as estimates of the volume of released urine and the duration of the release. The data can be stored in the memory 153b of the microcontroller 153 along with the bladder fullness data and the predicted urine release times described above.

The data regarding the actual urine release is used to adjust the prediction parameters used to predict releases of urine and thereby improve the accuracy of future predicted urine release times. This feedback mechanism allows the sensing apparatus 100 to self-calibrate to the individual bladder characteristics of the user and, as such, increases the reliability of the sensing apparatus 100.

In one implementation, the control unit 150 is configured to compare the actual urine release time detected by the urine sensor 190 with the predicted urine release time output by the microcontroller 153. The difference between the predicted urine release time and the actual detected urine release time is used to adjust the prediction parameters in order to increase the accuracy of future predictions. This adjustment can occur each time urine is detected by the urine sensor so that the prediction parameters are repeatedly refined. Each time the adjustment process is carried out; the new prediction parameters are stored in the memory 153b of the microcontroller 153 for use in predicting the time of the next release of urine. The repeated adjustment of the prediction parameters results in a statistical model of predictive accuracy being built in the memory 153b of the microcontroller 153. Therefore, generalized prediction parameters, for example based on bladder statistics for the average person, which were initially preloaded into the sensing unit 110 by the manufacturer are continuously updated and refined to improve the prediction of urine release times for a regular user of the sensing apparatus 100.

Adaptation of the prediction parameters can also take into account small movements in the location of the sensing unit 110 on the body which may affect the accuracy of the predicted releases of urine. The particular position of the sensing unit 110 relative to its position following the initialization process described above can be inferred by the microcontroller 153 from the ratios of the detected distance between the front and rear bladder walls at each piezoelectric crystal 110 location. The adaptation of the prediction parameters can be performed on data subsets corresponding to different sensing unit locations so that, if the most recently calculated ratios indicate that the sensing unit 110 has moved to a location which is known to provide unreliable data concerning the state of fullness of the bladder, the microcontroller 153 is configured to cause the alarm unit 180 to activate a subtle alarm to let the user or carer know that the sensing unit 110 should be repositioned on the body. Locations known to provide unreliable data may be locations in which the sensing unit 110 is detected as not overlapping, or only partially overlapping, the bladder. Unreliable data may also be caused by air trapped between the sensing unit 110 and the skin of the user affecting the acoustic coupling.

As the number of uses of the sensing unit 110 increases, so should the predictive accuracy of the unit 100 for a regular user. The predictive accuracy of the unit 100 can be defined in terms of a confidence level (%) that urine will be released in a specific time period, for example in a five minute window. As the predictive accuracy of the unit 100 increases and the unit 100 becomes better at predicting when urine will be released from a person's bladder, the confidence level for a specified time window will increase. As such, the control unit 150 may be configured so that a time window in which the alarm unit 180 warns that urine will be released in is shortened to provide a more accurate warning.

To give an example, when the sensing unit 110 is first used by a particular user, the prediction parameters referred to above are unlikely to be fully optimized for that user and, as such, prediction accuracy will be relatively low. It is therefore more likely under such circumstances that an alarm warning that urine will be released in the next five minutes will not be correct and that the predicted release of urine will not occur until after the five minute time frame has elapsed. This can be referred to as a false positive. Equally, it is also more likely under such circumstances that urine will be released before the warning alarm is activated. This is referred to as a false negative.

To increase the rate at which the prediction parameters are optimized for a particular user, the above-described adjustment of the prediction parameters can initially occur in a training mode of the apparatus 100 in which the alarm unit 180 does not output alarms warning of imminent releases urine. The training mode may be activated for a specified period of, for example, one day or one week of consistent use of the sensing apparatus 100. Once the sensing apparatus 100 has sufficiently self-calibrated to the user, the sensing apparatus 100 can be manually switched to a use mode, in which the alarm signals are activated. Alternatively, the sensing apparatus 100 may be configured to switch from the training mode to the use mode automatically after a predetermined number or urine releases have been detected and used to adapt the prediction parameters.

The user or carer can input an acceptable confidence level (e.g. 98%) to the sensing apparatus 100 upon which they wish the time window in which urination is predicted to occur, and thus the activation of the warning alarm, to be calculated. The user or carer may also input an acceptable percentage of false positives and/or false negatives (e.g. 5%) to reduce the length of the predicted time window if desired. Alternatively, an acceptable confidence level and percentage of false positives/negatives may be preloaded into the sensing unit 110 by the manufacturer.

When the sensing unit 110 is initially used, the time window in which urine is predicted to be released with a specified confidence level may be relatively long (e.g. five minutes). However, as the predictive accuracy of the apparatus 100 increases by adjustment of the prediction parameters (e.g. in the training mode), the time window in which urine is predicted to be released (for the same confidence level) will be significantly shorter (e.g. two minutes). The user can adjust the acceptable confidence level and number of false positives/negatives to meet their requirements. Alternatively, the confidence level and number of false positives/negatives may be automatically adjusted (e.g. respectively increased/decreased), for example by the algorithm, as the predictive accuracy of the sensing unit 110 increases.

The sensing unit 110 is hermetically sealed in a non-reactive, hypo-allergenic packaging which can be worn against the body for extended periods of time. For example, as shown in FIG. 1, the control unit 150 is encapsulated in a water and urine proof sealant material 200 to prevent it from getting wet and to protect it from accidental damage. The sealant material 200 may optionally be provided around the whole sensing unit 110. When the sensing unit 110 is worn, the sealant material 200 may directly contact the skin of the user over the bladder to act as an acoustic couplant. The material used for the sealant 200 may therefore be chosen so that it sticks directly to the skin with minimal air bubble interference and is comfortable for the user. The material 200 should preferably have some flexibility in its shape so that it can conform to the shape of the human body with which it is used. The material 200 should also preferably allow easy and effective cleaning and/or disinfecting to allow the unit 110 to conform to strict hygiene standards. A suitable material 200 is Sylgard 527 silicone rubber, which can be cleaned and disinfected by gentle rubbing whilst submerged in alcohol.

A principal use of the sensing apparatus 100 may be for reducing the toilet training period of a child by helping the child to associate the warning alarm and associated bladder feeling with a requirement to use the toilet. The sensing apparatus 100 may also be used by incontinent adults.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processing unit or other unit may fulfill the functions of several items recited in the claims. The mere fact that certain measures are recited in mutually different dependent claims does not indicate that a combination of these measures cannot be used to an advantage. A computer program may be stored/distributed on a suitable medium, such as an optical storage medium or a solid-state medium supplied together with or as part of other hardware, but may also be distributed in other forms, such as via the Internet or other wired or wireless telecommunications systems. Any reference signs in the claims should not be construed as limiting the scope.

1. A sensing apparatus configured to:
predict a release of urine using one or more prediction parameters;
detect an actual release of urine; and
adapt at least one of the one or more prediction parameters based on the actual release of urine to increase prediction accuracy.

2. An apparatus according to claim 1, configured to adapt the at least one prediction parameter based on a difference between the predicted release of urine and the actual release of urine.

3. An apparatus according to claim 1, configured to:
predict the release of urine by predicting a time at which the release of urine will occur;
detect the actual release of urine by detecting a time at which the actual release of urine occurs; and
adapt the at least one prediction parameter based on a time difference between the predicted time of urine release and the detected time of urine release.

4. An apparatus according to claim 1, configured to make intermittent estimates of a state of fullness of a bladder and to predict the release of urine using the state of fullness estimates and the prediction parameters.

5. An apparatus according to claim 5, configured to enter a power-saving mode between the intermittent state of fullness estimates.

6. An apparatus according to claim 4, configured to estimate the state of fullness of the bladder by:
emitting an ultrasound wave towards the bladder;
detecting an ultrasound wave reflected back from a wall of the bladder; and
analyzing the reflected ultrasound wave to estimate the state of fullness of the bladder.

7. An apparatus according to claim 6, comprising:
a plurality of ultrasound transducer elements for emitting and detecting the ultrasound waves;
driving and receiving circuitry configured to respectively cause the ultrasound transducer elements to emit the ultrasound waves and to detect the ultrasound waves in the ultrasound transducer elements; and
a controller configured to estimate the state of fullness of the bladder and to predict the release of urine based on the estimated state of fullness of the bladder and the prediction parameters.

8. An apparatus according to claim 1, configured to adapt at least one of the one or more prediction parameters based on the position of the sensing apparatus relative to a bladder.

9. An apparatus according to claim 1, comprising an alarm unit configured to provide an alert warning of a predicted release of urine.

10. An apparatus according to claim 1, comprising one or more indicators configured to indicate when the sensing apparatus is correctly located in a position overlapping a bladder.

11. An apparatus according to claim 1 comprising a sensing unit for wearing close to the skin of a user and an external unit configured to communicate with the sensing unit for predicting the release of urine.

12. An item of clothing comprising at least part of the sensing apparatus according to claim 1.

13. A method for warning of a release of urine comprising:
predicting a release of urine using one or more prediction parameters;
detecting an actual release of urine; and
adapting at least one of the one or more prediction parameters based on the actual release of urine to increase prediction accuracy.

14. A method according to claim 13, wherein:
predicting a release of urine comprises predicting a time at which the release of urine will occur;
detecting the actual release of urine comprises detecting a time at which the actual release of urine occurs; and
adapting the at least one prediction parameter comprises adapting the at least one prediction parameter based on a time difference between the predicted time of urine release and the detected time of urine release.

15. A computer program which, when executed by a processor, causes the processor to perform a method according to claim 13.
